(19) United States
(12) Patent Application Publication
(10) Pub. No.: US 2004/0106605 A1
(43) Pub. Date: Jun. 3, 2004

(54) Synergistic methods and compositions for treating cancer

(76) Inventors: Joan M. Carboni, Yardley, PA (US); Warren W. Hurlburt, Mount Laurel, NJ (US); Marco M. Gottardis, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/676,214

(22) Filed: Oct. 1, 2003

Related U.S. Application Data
(60) Provisional application No. 60/415,416, filed on Oct. 2, 2002.

(51) Int. Cl. A61K 31/54; A61K 31/513; A61K 31/4439

(52) U.S. Cl. 514/226.8; 514/228.2; 514/269; 514/338

(57) ABSTRACT

The use of IGF1R inhibitors in combination with EGFR inhibitors are described for the synergistic treatment of cancer.

### RELATED APPLICATIONS

This application claims priority benefit under Title 35 § 119(e) of U.S. Provisional Application No. 60/415,416, filed Oct. 2, 2002, entitled “Synergistic Methods and Compositions for Treating Cancer.”

### FIELD OF THE INVENTION

The present invention relates to therapies for the treatment of cancer, specifically to synergistic methods for treating cancer using IGF1R inhibitors in combination with EGFR inhibitors.

### BACKGROUND OF THE INVENTION

Chemotherapy, the systemic administration of antineoplastic agents that travel throughout the body via the blood circulatory system, along with and often in conjunction with surgery and/or radiation treatment, has for years been widely utilized in the treatment of a wide variety of cancers.

Today, there are a variety of antineoplastic agents that have successfully been used in the treatment of cancer. However, the search continues for more efficacious and less toxic agents.

Tyrosine kinases are a class of enzymes that have proven to be useful agents for the treatment of cancer. Tyrosine kinases catalyze the transfer of the terminal phosphate of adenosine triphosphate to the phenolic hydroxyl group of a tyrosine residue present in the target protein. Tyrosine kinases play a critical role in signal transduction for several cellular functions including cell proliferation, carcinogenesis, apoptosis, and cell differentiation (Plowman, G. D.; Ullrich, A.; Shawver, L. K.: Receptor Tyrosine Kinases As Targets For Drug Intervention. DN&P (1994) 7: 334-339). Inhibitors of these enzymes are actually useful for the treatment or prevention of a variety of proliferative diseases that are dependent on these enzymes. Strong epidemiologic evidence suggests that the overexpression or activation of receptor protein tyrosine kinases leading to constitutive mitogenic signaling is an important factor in a growing number of human malignancies. Tyrosine kinases that have been implicated in these processes include Abl, CDK's, EGF, EMT, FGF, FAK, Flk-1/KDR, HER-2, IGF-1R, IR, LCK, MET, PDGF, Src, and VEGF (Traxler, P. M. Protein Tyrosine Kinase Inhibitors in Cancer Treatment. Exp. Opin. Ther. Patents (1997) 7: 571-588; incorporated herein by reference).

The IGF1R (insulin-like growth factor-1 receptor) affects cell mitogenesis, survival, transformation, and insulin-like activities by the binding of its ligands, IGF1 and IGF2. This receptor influences post natal growth physiology, and its activity has been associated with malignant disorders such as breast cancer. See, Ellis et al., Breast Cancer Res. Treat. 1998, 52, 175. The anti-apoptotic effect induced by the IGF1/IGF1R system correlates to the induction of chemoresistance in various tumors. See, Grothey et al., J. Cancer Res. Clin. Oncol., 1999, 125, 166-73. Accordingly, inhibitors of IGF1R are useful in the treatment of cancer, as evidenced in U.S. patent application Ser. No. 10/105,599. IGF1R inhibitors are useful as single agents and also in combination with other anticancer agents.

Expression of EGFR is common in many solid tumors, such as colorectal and lung carcinomas as well as cancers of the head and neck. It correlates with increased metastasis, decreased survival and a poor prognosis. EGFR protects malignant tumour cells from the cytotoxic effects of chemotherapy and radiotherapy, making these treatments less effective.

However, although combination chemotherapy has improved the response and survival rates of patients with hematological malignancies and some solid tumors, it is well known that anti-cancer drugs often bring on serious side effects that limit the doses physicians can administer. Synergistic combination chemotherapy is especially desirable because the synergy between active ingredients allows for the use of smaller doses of one or both active ingredients, provides greater efficacy at the same doses, and/or prevents or delays the build-up of multi-drug resistance. Accordingly, there is a need in the art for synergistic chemotherapy regimens that are effective for the treatment of cancer with improved toxicity profiles.

### SUMMARY OF THE INVENTION

It has now been found, and this forms the subject of the present invention, that the efficacy of both IGF1R inhibitors and EGFR inhibitors are considerably improved when they are administered in combination, resulting in methods for the synergistic treatment of cancer.

### BRIEF DESCRIPTION OF THE DRAWINGS

FIG. 1 is an isobologram depicting the synergistic anticancer activity achieved when an IGF1R inhibitor (Compound 1) is administered in combination with an EGFR inhibitor, gefitinib, in IGF1R sal cells.

FIG. 2 is an isobologram depicting the synergistic anticancer activity achieved when an IGF1R inhibitor (Compound 1) is administered in combination with an EGFR inhibitor, gefinitib, in MCF-7 cells.

FIG. 3 is an isobologram depicting the synergistic anticancer activity achieved when an IGF1R inhibitor (Compound 1) is administered in combination with an EGFR inhibitor, gefitinib, in MDA-Pca-2b cells.

FIG. 4 is an isobologram depicting the synergistic anticancer activity achieved when an IGF1R inhibitor (Compound 1) is administered in combination with an EGFR inhibitor, cetuximab, in GEO cells.

FIG. 5 is an is an isobologram depicting the synergistic anticancer activity achieved when an IGF1R inhibitor (Compound 2) is administered in combination with an EGFR inhibitor, cetuximab, in GEO cells.

FIG. 6 is an isobologram depicting the synergistic anticancer actibity achieved when an IGF1R inhibitor (Compound 2) is administered in combination with an EGFR inhibitor, gefitinib in RD1 cells.

FIG. 7 is an isobologram depicting the synergistic anticancer activity achieved when an IGF1R inhibitor (Compound 1) is administered in combination with an EGFR inhibitor, erlotinib, in MDA-Pca-2b cells.

FIG. 8 is an isobologram depicting the synergistic anticancer activity achieved when an IGF1R inhibitor (Compound 1) is administered in combination with an EGFR inhibitor, erlotinib, in MCF 7 cells.

FIG. 9 shows the effects of an IGFR inhibitor (Compound 1) and an EGFR inhibitor, cetuximab, singly or in combination, on the growth of the GEO human colon carcinoma xenograft model in nude mice.

### DETAILED DESCRIPTION

Advantageously, the present invention provides a method for the synergistic treatment of cancer comprising administering a synergistically, therapeutically effective amount of (1) an IGF1R inhibitor and (2) an EGFR inhibitor to a mammalian species, preferably a human, in need thereof.

As used herein, the term “synergistic” means that the effect achieved with the methods and compositions of this invention is greater than the sum of the effects that results from methods and compositions comprising EGFR inhibitors and IGF1R inhibitors separately.

Further advantages over previously disclosed methods include the ability of the instant combination of IGF1R inhibitors and the EGFR inhibitor to be individually varied depending on the nature of the cancer cells to be treated. It is also anticipated that the therapeutic effect of the instant compositions may be achieved with smaller amounts of either inhibitor than would be required if such inhibitors were administered alone. This approach minimizes any non-mechanism-based adverse toxicity effects which might result from administration of an amount of an EGFR inhibitor or an IGF 1 R inhibitor alone sufficient to achieve the same therapeutic effect.

The present invention provides methods for the synergistic treatment of a variety of cancers, including, but not limited to, the following:

carcinoma including that of the bladder (including accelerated and metastatic bladder cancer), breast, cervical, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);

hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma;

hematopoietic tumors of myeloid lineage including acute and chronic myelogenous leukemias, myelodysplastic syndrome, myeloid leukemia, and promyelocytic leukemia;

tumors of the central and peripheral nervous system including astrocytoma, neuroblastoma, glioma, and schwannomas;

tumors of mesenchymal origin including fibrosarcoma, liposarcoma, rhabdomyosarcoma, and osteosarcoma; and

other tumors including melanoma, xenoderma pigmentosum, keratoactanthoma, seminoma, thyroid follicular cancer, and teratocarcinoma.

In a preferred embodiment of this invention, a method is provided for the synergistic treatment of cancerous tumors. The synergistic method of this invention reduces the development of tumors, reduces tumor burden, or produces tumor regression in a mammalian host.

As used herein, the term “IGF1R inhibitor” refers to any biological or small molecule that inhibits the activity of the IGF1 receptor, thereby providing an anti-cancer effect.

IGF1R inhibitors of the present invention and methods for making them are described in U.S. application Ser. No. 10/263,448, the disclosure of which is herein incorporated by reference in its entirety. Additional IGF1R inhibitors that are useful in the present invention include those described by U.S. Patent Application 60/437,926; U.S. Patent Application 60/415,066; WO03/048133; WO 01/25220; U.S. Pat. No. 6,337,338 (WO 00/35455); WO 02/102804; WO 02/092599; WO 03/024967; WO 03/035619; WO 03/035616; and WO 03/018022, the disclosures of which are herein incorporated by reference in their entirety.

In some embodiments of the present invention, the IGF1R inhibitor has the formula I:

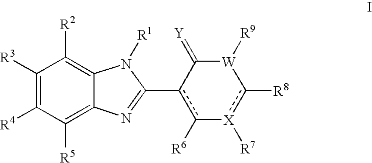

and includes its enantiomers, diastereomers, pharmaceutically acceptable salts, hydrates, prodrugs and solvates thereof;

wherein

X is N, C or a direct bond;

Y is O or S;

W is N, C, O, or S; provided that if W is O or S, R⁹ is absent;

R¹ is H, alkyl, or alkoxy;

R² and R⁹ are independently H or alkyl;

R³ is H, C₁₋₆ alkyl, alkenyl, alkynyl, cycloalkyl, heterocycloalkyl, halo, amino, —OR⁶⁰, —NO₂, —OH, —SR⁶⁰, —NR⁶⁰R⁶¹, —CN, —C(O)R⁶⁰, —CO₂R⁶, —CONR⁶⁰R⁶¹, OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3:

R⁴ is H, halo, alkyl or haloalkyl;

R⁵ is H, alkyl, halo, or aryl;

R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;

R⁶⁰, R⁶¹, R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;

R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹, heteroaryl or heterocycloalkyl; and

R³⁰ and R³¹ are, independently, hydrogen, alkyl, or cycloalkyl.

In some embodiments of the present invention, R¹ is H, alkyl or alkoxy, R² is H; R³ is H, alkyl, —CN, halo, —C(O)R⁶⁰—C(O)NR⁶⁰R⁶¹, —S(O)₂R⁶³, piperazine, piperidine, morpholine, triazole, imidazole, wherein the piperazine, piperidine, morpholine, triazole, or imidazole is substituted with H, alkyl, —NHC(O)alkyl, —NHC(O)₂alkyl, —NHC(O)alkoxy, —O—(CH₂)ₙR⁶⁴ wherein R⁶⁴ is hydroxy, alkoxy, morpholine, or tetrahydropyrimidine; and R⁶ is —NH-Z-phenyl; —NH-Z-imidazole; or —NH-Z-pyrazole wherein Z is C1 to C2 alkyl.

In some embodiments of the present invention, the IGF1R inhibitor is selected from the group consisting of:

(S)-4-(2-Hydroxy-1-phenyl-ethylamino)-3-(6-imidazol-1-yl-4-methyl-1H-benzimidazol-2-yl) 1H-pyridin-2-one;

(±)-4-[2-Hydroxy-2-(3-iodo-phenyl)-ethylamino]-3-(6-imidazol-1-yl-4-methyl-1H-benzimidazol-2-yl)-1H-pyridin-2-one;

(±)-4-[2-(3-Chloro-phenyl)-2-hydroxy-ethylamino]-3-(6-imidazol-1-yl-4-methyl-1H-benzimidazol-2-yl)-1H-pyridin-2-one;

(±)-4-[2-(3-Bromo-phenyl)-2-hydroxy-ethylamino]-3-(6-imidazol-1-yl-4-methyl-1H-benzimidazol-2-yl)-1H-pyridin-2-one;

(S)-4-[2-(2-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(3-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(4-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(2-Bromo-phenyl)-1-hydroxymethyl-ethylamino]-3-(6-imidazol-1-yl-4-methyl-1H-benzimidazol-2-yl)-1H-pyridin-2-one;

(S)-4-[2-(3-Bromo-phenyl)-1-hydroxymethyl-ethylamino]-3-(6-imidazol-1-yl-4-methyl-1H-benzimidazol-2-yl)-1H-pyridin-2-one;

(±)-4-(1-Hydroxymethyl-2-pentafluorophenyl-ethylamino)-3-(6-imidazol-1-yl-4-methyl-1H-benzimidazol-2-yl)-1H-pyridin-2-one;

(S)-4-(1-Hydroxymethyl-2-pyridin-4-yl-ethylamino)-3-(6-imidazol-1-yl-4-methyl-1H-benzimidazol-2-yl)-1H-pyridin-2-one;

(S)-4-[1-Hydroxymethyl-2-(2-naphthalenyl)-ethylamino]-3-(6-imidazol-1-yl-4-methyl-1H-benzimidazol-2-yl)-1H-pyridin-2-one;

3-(6-Imidazol-1-yl-4-methyl-1H-benzimidazol-2-yl)-4-(pyridin-2-ylmethoxy)-1H-pyridin-2-one;

(±)-4-[2-(3-Bromo-phenyl)-2-fluoro-ethylamino]-3-(6-imidazol-1-yl-4-methyl-1H-benzimidazol-2-yl)-1H-pyridin-2-one;

(S)-2-[4-(1-Hydroxymethyl-2-phenyl-ethylamino)-2-oxo-1,2-dihydro-pyridin-3-yl]-7-methyl-3H-benzimidazole-5-carbonitrile;

(±)-2-{4-[2-(3-Chloro-phenyl)-2-hydroxy-ethylamino]-2-oxo-1,2-dihydro-pyridin-3-yl}-7-methyl-3H-benzimidazole-5-carbonitrile;

(S)-2-{4-[2-(3-Chloro-phenyl)-1-hydroxymethyl-ethylamino]-2-oxo-1,2-dihydro-pyridin-3-yl}-7-methyl-3H-benzimidazole-5-carbonitrile;

(±)-2-{4-[2-(3-Bromo-4-methoxy-phenyl)-2-hydroxy-ethylamino]-2-oxo-1,2-dihydro-pyridin-3-yl}-7-methyl-3H-benzimidazole-5-carbonitrile;

(±)-2-{4-[2-(3-Fluoro-phenyl)-2-hydroxy-ethylamino]-2-oxo-1,2-dihydro-pyridin-3-yl}-7-methyl-3H-benzimidazole-5-carbonitrile;

(±)-2-{4-[2-(3-Bromo-phenyl)-2-hydroxy-ethylamino]-2-oxo-1,2-dihydro-pyridin-3-yl}-7-methyl-3H-benzimidazole-5-carbonitrile;

(S)-2-[4-(2-Hydroxy-2-phenyl-ethylamino)-2-oxo-1,2-dihydro-pyridin-3-yl]-7-methyl-3H-benzimidazole-5-carbonitrile;

(±)-3-(1H-Benzimidazol-2-yl)-4-[2-(3-bromo-phenyl)-2-hydroxy-ethylamino]-1H-pyridin-2-one;

(S)-3-(1H-Benzimidazol-2-yl)-4-(1-hydroxymethyl-2-phenyl-ethylamino)-1H-pyridin-2-one;

(±)-3-(1H-Benzimidazol-2-yl)-4-[2-(3-bromo-4-methoxy-phenyl)-2-hydroxy-ethylamino]-1H-pyridin-2-one;

(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid isopropylamide;

(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid ethylamide;

(S)-4-(1-Hydroxymethyl-2-phenyl-ethylamino)-3-{4-methyl-6-[4-(1-phenyl-methanoyl)piperazin-1-yl]-1H-benzimidazol-2-yl}-1H-pyridin-2-one;

(S)-4-(1-Hydroxymethyl-2-phenyl-ethylamino)-3-[6-(4-isopropyl-piperazin-1-yl)-4-methyl-1H-benzimidazol-2-yl]-1H-pyridin-2-one;

(S)-3-[6-(4-Benzyl-piperazine-1-yl)-4-methyl-1H-benzimidazol-2-yl]-4-(1-hydroxymethyl-2-phenyl-ethylamino)-1H-pyridin-2-one;

(±)-3-[6-(4-Acetyl-piperazine-1-yl)-4-methyl-1H-benzimidazol-2-yl]-4-[2-(3-chloro-phenyl)-2-hydroxy-ethylamino]-1H-pyridin-2-one;

(±)-4-[2-(3-Chloro-phenyl)-2-hydroxy-ethylamino]-3-(4-methyl-6-piperazin-1-yl-1H-benzimidazol-2-yl)-1H-pyridin-2-one;

(±)-4-[2-(3-Chloro-phenyl)-2-hydroxy-ethylamino]-3-[6-(4-isopropyl-piperazine-1-yl)-4-methyl-1 H-benzimidazol-2-yl]-1H-pyridin-2-one;

(S)-6-(1-Hydroxymethyl-2-phenyl-ethylamino)-5-(6-imidazol-1-yl-4-methyl-1H-benzimidazol-2-yl)-3H-pyrimidin-4-one;

(S)-2-[6-Chloro-5-(6-imidazol-1-yl-4-methyl-1H-benzimidazol-2-yl)-pyrimidin-4-ylamino]-3-phenyl-propan-1-ol;

(S)-4-(2-Hydroxy-2-phenyl-ethylamino)-3-(6-imidazol-1-yl-4-methyl-1H-benzimidazol-2-yl)-1H-pyridin-2-one;

(R)-4-(2-Hydroxy-2-phenyl-ethylamino)-3-(6-imidazol-1-yl-4-methyl-1H-benzimidazol-2-yl)-1H-pyridin-2-one;

(1S,2R)-4-(1-Hydroxy-indan-2-ylamino)-3-(6-imidazol-1-yl-4-methyl-1H-benzimidazol-2-yl)-1H-pyridin-2-one;

(±)-4-[2-Hydroxy-2-(3-hydroxy-phenyl)-ethylamino]-3-(6-imidazol-1-yl-4-methyl-1H-benzimidazol-2-yl)-1H-pyridin-2-one;

(S)-4-(2-Hydroxy-2-pyridin-2-yl-ethylamino)-3-(6-imidazol-1-yl-4-methyl-1H-benzimidazol-2-yl)-1H-pyridin-2-one;

(±)-N-(3-{1-Hydroxy-2-[3-(6-imidazol-1-yl-4-methyl-1H-benzimidazol-2-yl)-2-oxo-1,2-dihydro-pyridin-4-ylamino]-ethyl}-phenyl)-methanesulfonamide;

(±)-4-[2-(3-Fluoro-phenyl)-2-hydroxy-ethylamino]-3-(6-imidazol-1-yl-4-methyl-1H-benzimidazol-2-yl)-1H-pyridin-2-one;

(±)-4-[2-(3-Chloro-4-fluoro-phenyl)-2-hydroxy-ethylamino]-3-(6-imidazol-1-yl-4-methyl-1H-benzimidazol-2-yl)-1H-pyridin-2-one;

(S)-4-[2-(3-Fluoro-phenyl)-1-hydroxymethyl-ethylamino]-3-(6-imidazol-1-yl-4-methyl-1H-benzimidazol-2-yl)-1H-pyridin-2-one;

(±)-4-[2-(3-Chloro-phenyl)-2-hydroxy-ethylamino]-3-(6-imidazol-1-yl-1H-benzimidazol-2-yl)-1H-pyridin-2-one;

(±)-4-[2-(3-Bromo-4-methoxy-phenyl)-2-hydroxy-ethylamino]-3-(6-imidazol-1-yl-4-methyl-1H-benzimidazol-2-yl)-1H-pyridin-2-one;

(S)-4-[2-(3-Bromo-phenyl)-2-hydroxy-ethylamino]-3-(6-imidazol-1-yl-4-methyl-1H-benzimidazol-2-yl)-1H-pyridin-2-one;

(S)-4-[2-(3-Chloro-phenyl)-2-hydroxy-ethylamino]-3-(6-imidazol-1-yl-4-methyl-1H-benzimidazol-2-yl)-1H-pyridin-2-one;

(R)-4-[2-(3-Chloro-phenyl)-2-hydroxy-ethylamino]-3-(6-imidazol-1-yl-4-methyl-1H-benzimidazol-2-yl)-1H-pyridin-2-one;

(±)-4-[2-(3-Chloro-4-methoxy-phenyl)-2-hydroxy-ethylamino]-3-(6-imidazol-1-yl-4-methyl-1H-benzimidazol-2-yl)-1H-pyridin-2-one;

(±)-(2-Chloro-4-{1-hydroxy-2-[3-(6-imidazol-1-yl-4-methyl-1H-benzimidazol-2-yl)-2-oxo-1,2-dihydro-pyridin-4-ylamino]-ethyl}-phenyl)-carbamic acid methyl ester;

(S)-4-(1-Hydroxymethyl-2-phenyl-ethylamino)-3-[4-methyl-6-(4-methyl-piperazin-1-yl)-1H-benzimidazol-2-yl]-1H-pyridin-2-one;

(S)-4-(1-Hydroxymethyl-2-phenyl-ethylamino)-3-[4-methyl-6-(4-n-butyl-piperazin-1-yl)-1H-benzimidazol-2-yl]-1H-pyridin-2-one;

(S)-3-{6-[4-(2-Hydroxy-ethyl)-piperazin-1-yl]-4-methyl-1H-benzimidazol-2-yl}-4-(1-hydroxymethyl-2-phenyl-ethylamino)-1H-pyridin-2-one;

(S)-4-{2-[4-(1-Hydroxymethyl-2-phenyl-ethylamino)-2-oxo-1,2-dihydro-pyridin-3-yl]-7-methyl-3H-benzimidazol-5-yl}-piperazine-1-carboxylic acid amide;

(±)-4-[2-(3-Chloro-phenyl)-2-hydroxy-ethylamino]-3-(4-methyl-6-piperazin-1-yl-1H-benzimidazol-2-yl)-1H-pyridin-2-one;

(±)-4-[2-(3-Chloro-phenyl)-2-hydroxy-ethylamino]-3-[6-(4-ethyl-piperazin-1-yl)-4-methyl-1H-benzimidazol-2-yl]-1H-pyridin-2-one;

(±)-4-[2-(3-Chloro-phenyl)-2-hydroxy-ethylamino]-3-{6-[4-(2-hydroxy-ethyl)piperazin-1-yl]-4-methyl-1H-benzimidazol-2-yl}-1 H-pyridin-2-one;

(±)-4-[2-(3-Chloro-phenyl)-2-hydroxy-ethylamino]-3-(4-methyl-6-morpholin-4-yl-1H-benzimidazol-2-yl)-1H-pyridin-2-one;

(±)-4-[2-(3-Bromo-phenyl)-2-hydroxy-ethylamino]-3-(4-methyl-6-morpholin-4-yl-1H-benzimidazol-2-yl)-1H-;

(±)-4-[2-(3-Bromo-4-methoxy-phenyl)-2-hydroxy-ethylamino]-3-(4-methyl-6-morpholin-4-yl-1H-benzimidazol-2-yl)-1H-pyridin-2-one;

(±)-4-[2-(3-Bromo-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

(±)-4-[2-(3-Bromo-phenyl)-2-hydroxy-ethylamino]-3-(4-methyl-6-piperazin-1-yl-1H-benzimidazol-2-yl}-1H-pyridin-2-one;

(±)-4-[2-(3-Bromo-phenyl)-2-hydroxy-ethylamino]-3-(4-methyl-6-piperazin-1-yl-1H-benzimidazol-2-yl}-1H-pyridin-2-one;

(±)-3-[6-(4-Acetyl-piperazin-1-yl)-4-methyl-1H-benzimidazol-2-yl]-4-[2-(3-bromo-phenyl)-2-hydroxy-ethylamino]-1H-pyridin-2-one;

(S)-4-(1-hydroxymethyl-2-phenyl-ethylamino)-3-[4-methyl-6-(2-morpholin-4-yl-ethylamino)-1H-benzimidazol-2-yl]-1H-pyridin-2-one;

(±)-6-[2-(3-Chloro-phenyl)-2-hydroxy-ethylamino]-5-(6-imidazol-1-yl-4-methyl-1H-benzimidazol-2-yl)-3H-pyrimidin-4-one;

(±)-4-[2-(3-Chloro-phenyl)-2-hydroxy-ethylamino]-3-[6-(1-hydroxy-1-methyl-ethyl)-4-methyl-1H-benzimidazol-2-yl]-1H-pyridin-2-one;

(±)-3-(6-Aminomethyl-4-methyl-1H-benzimidazol-2-yl)-4-[2-(3-chloro-phenyl)-2-hydroxy-ethylamino]-1H-pyridin-2-one;

(±)-4-[2-(3-Chloro-phenyl)-2-hydroxy-ethylamino]-3-(6-hydroxymethyl-4-methyl-1H-benzimidazol-2-yl)-1H-pyridin-2-one;

(S)-4-(1-Benzyl-2-hydroxy-ethylamino)-3-(4-methyl-6-morpholin-4-yl-1H-benzimidazol-2-yl)-1H-pyridin-2-one; and

(S)-4-(1-Benzyl-2-hydroxy-ethylamino)-3-(4-methyl-6-piperidin-1-yl-1H-benzimidazol-2-yl)-1H-pyridin-2-one;

(S)-4-(1-Benzyl-2-hydroxy-ethylamino)-3-(4-methyl-6-piperidin-1-yl-1H-benzimidazol-2-yl)-1H-pyridin-2-one;

4-[2-(3-Chloro-4-methylsulfanyl-phenyl)-2-hydroxy-ethylamino]-3-(4-methyl-6-piperazin-1-yl-1H-benzoimidazol-2-yl)-1H-pyridin-2-one;

4-[2-(3-Chloro-4-fluoro-phenyl)-2-hydroxy-ethylamino]-3-(4-methyl-6-piperazin-1-yl-1H-benzoimidazol-2-yl)-1H-pyridin-2-one;

3-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-propionitrile;

4-[2-(3-Chloro-phenyl)-2-hydroxy-ethylamino]-3-{6-[4-(2-methanesulfonyl-ethyl)-piperazin-1-yl]-4-methyl-1H-benzoimidazol-2-yl}-1H-pyridin-2-one;

3-[4-(2-{4-[2-(3-Bromo-4-methoxy-phenyl)-2-hydroxy-ethylamino]-2-oxo-1,2-dihydro-pyridin-3-yl}-3H-benzoimidazol-5-yl)-7-methyl-piperazin-1-yl]-propionitrile;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-fluoro-ethyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-methoxy-ethyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid prop-2-ynyl ester;

4-(2-{4-[2-(3-Bromo-4-methoxy-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;

(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carboxylic acid ethyl ester;

4-[2-(3-Chloro-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoro-ethyl)-piperazin-1-yl]-4-methyl-1H-benzoimidazol-2-yl}-1H-pyridin-2-one;

4-[2-(3-Chloro-4-fluoro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Bromo-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(3,3,3-trifluoro-propyl)-piperazin-1-yl]-1H-benzoimidazol-2-yl}-1H-pyridin-2-one;

4-[2-(3-Chloro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(3,4,4-trifluoro-but-3-enyl)-piperazin-1-yl]-1H-benzoimidazol-2-yl}-1H-pyridin-2-one;

4-[2-(3-Chloro-phenyl)-2-hydroxy-ethylamino]-3-{6-[4-(3-fluoro-2-hydroxy-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-hydroxy-2-methyl-propyl)-piperazin-1-yl]-4-methyl-1H-benzoimidazol-2-yl}-1H-pyridin-2-one;

(S)-4-[2-(3-Chloro-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

(S)-4-[2-(3-Bromo-4-methoxy-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetonitrile;

4-[2-(3-Chloro-phenyl)-2-hydroxy-ethylamino]-3-{6-[4-(4-fluoro-butyryl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2,2-difluoro-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methanesulfonyl-acetyl)-piperazin-1-yl]-4-methyl-1H-benzoimidazol-2-yl}-1H-pyridin-2-one;

3-[6-(4-Acetyl-piperazin-1-yl)-4-methyl-1H-benzoimidazol-2-yl]-4-[2-(3-chloro-phenyl)-2-hydroxy-ethylamino]-1H-pyridin-2-one;

4-[2-(3-Chloro-phenyl)-2-hydroxy-ethylamino]-3-(4-methyl-6-{4-[2-(1-oxo-114-thiomorpholin-4-yl)-acetyl]-piperazin-1-yl}-1H-benzoimidazol-2-yl)-1H-pyridin-2-one;

4-[2-(3-Chloro-phenyl)-2-hydroxy-ethylamino]-3-(6-{4-[2-(11,1-dioxo-116-thiomorpholin-4-yl)-acetyl]-piperazin-1-yl}-4-methyl-1H-benzoimidazol-2-yl)-1H-pyridin-2-one;

4-[2-(3-Chloro-phenyl)-2-hydroxy-ethylamino]-3-{4-methyl-6-[4-(2-thiomorpholin-4-yl-acetyl)-piperazin-1-yl]-1H-benzoimidazol-2-yl}-1H-pyridin-2-one;

4-[2-(3-Chloro-phenyl)-2-hydroxy-ethylamino]-3-{6-[4-(2-methanesulfinyl-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methoxy-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(2-methylsulfanyl-acetyl)-piperazin-1-yl]-1H-benzoimidazol-2-yl}-1H-pyridin-2-one;

3-{6-[4-(2-Chloro-acetyl)-piperazin-1-yl]-4-methyl-1H-benzoimidazol-2-yl}-4-[2-(3-chloro-phenyl)-2-hydroxy-ethylamino]-1H-pyridin-2-one;

(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carbaldehyde;

(S)-4-(2-{4-[2-(3-Chloro-phenyl)-2-hydroxy-ethylamino]-2-oxo-1,2-dihydro-pyridin-3-yl}-7-methyl-3H-benzimidazol-5-yl)-piperazine-1-carbaldehyde;

(S)-4-[2-(3-Chloro-phenyl)-2-hydroxy-ethylamino]-3-(4-methyl-6-morpholin-4-yl-1H-benzoimidazol-2-yl)-1H-pyridin-2-one;

4-[2-(3-Bromo-4-methoxy-phenyl)-2-hydroxy-ethylamino]-3-(4-methyl-6-morpholin-4-yl-1H-benzoimidazol-2-yl)-1H-pyridin-2-one;

4-[2-(3-Chloro-4-fluoro-phenyl)-2-hydroxy-ethylamino]-3-(4-methyl-6-morpholin-4-yl-1H-benzoimidazol-2-yl)-1H-pyridin-2-one;

4-[2-(3-Chloro-4-methoxy-phenyl)-2-hydroxy-ethylamino]-3-(4-methyl-6-morpholin-4-yl-1H-benzoimidazol-2-yl)-1H-pyridin-2-one;

4-[2-(7-Bromo-2,3-dihydro-benzofuran-5-yl)-2-hydroxy-ethylamino]-3-(4-methyl-6-morpholin-4-yl-1H-benzoimidazol-2-yl)-1H-pyridin-2-one;

4-[2-(3-Chloro-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;

4-[2-(3-Bromo-4-methoxy-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;

4-[2-(3-Chloro-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(7-Bromo-2,3-dihydro-benzofuran-4-yl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(7-bromo-2,3-dihydro-benzofuran-4-yl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methoxy-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;

4-[2-(3-Chloro-phenyl)-2-(S)-hydroxy-ethylamino]-3-[4-methyl-6-(4-methyl-piperazin-1-yl)-1H-benzoimidazol-2-yl]-1H-pyridine-2-one;

4-[2-(3-Bromo-4-methoxy-phenyl)-2-(S)-hydroxy-ethylamino]-3-[4-methyl-6-(4-methyl-piperazin-1-yl)-1H-benzoimidazol-2-yl]-1H-pyridine-2-one;

4-[2-(3-Chloro-phenyl)-2-hydroxy-ethylamino]-3-{6-[4-(acetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;

4-[2-(3-Bromo-4-methoxy-phenyl)-2-hydroxy-ethylamino]-3-{6-[4-(acetamido)-piperidin-1-yl]-4-methyl-1H-benzoimidazol-2-yl}-1H-pyridin-2-one;

4-[2-(3-Bromo-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methoxyethoxycarbamoyl)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(methoxycarbamoyl)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroethoxy carbamoyl)-piperidin-1-yl]-4-methyl-1H-benzoimidazol-2-yl}-1H-pyridin-2-one;

(S)-4-[2-(3-Chloro-phenyl)-2-hydroxy-ethylamino]-3-[4-methyl-6-(2-morpholin-4-yl-ethoxy)-1H-benzimidazol-2-yl]-1H-pyridin-2-one;

(S)-4-[2-(3-Bromo-4-methoxy-phenyl)-2-hydroxy-ethylamino]-3-[4-methyl-6-(2-morpholin-4-yl-ethoxy)-1H-benzimidazol-2-yl]-H-pyridin-2-one;

(S)-4-[2-(3-Chloro-phenyl)-2-hydroxy-ethylamino]-3-[4-methyl-6-(2-methoxy-ethoxy)-1H-benzimidazol-2-yl]-1H-pyridin-2-one;

(S)-4-[2-(3-Chloro-phenyl)-2-hydroxy-ethylamino]-3-[4-methyl-6-(2-hydroxy-ethoxy)-1H-benzimidazol-2-yl]-1H-pyridin-2-one;

(S)-4-[2-(3-Bromo-4-methoxy-phenyl)-2-hydroxy-ethylamino]-3-[4-methyl-6-(2-morpholin-4-yl-propoxy)-1H-benzimidazol-2-yl]-1H-pyridin-2-one;

(S)-4-[2-(3-Chloro-phenyl)-2-hydroxy-ethylamino]-3-[4-methyl-6-(2-morpholin-4-yl-propoxy)-1H-benzimidazol-2-yl]-1H-pyridin-2-one;

(S)-3-(4-Bromo-6-morpholin-4-ylmethyl-1H-benzimidazol-2-yl)-4-[2-(3-chloro-phenyl)-2-hydroxy-ethylamino]-1H-pyridin-2-one;

(S)-3-[4-Bromo-6-(4-methyl-piperazin-1-ylmethyl-1H-benzimidazol-2-yl)-4-[2-(3-chloro-phenyl)-2-hydroxy-ethylamino]-1H-pyridin-2-one;

(S)-4-[2-(3-Chloro-phenyl)-2-hydroxy-ethylamino]-3-[4-methyl-6-(4-methyl-piperazin-1-ylmethyl)-1H-benzimidazol-2-yl]-1H-pyridin-2-one;

4-[2-(3-Chloro-phenyl)-2-(S)-hydroxy-ethylamino]-3-[4-methyl-6-(1,4,5,6-tetrahydropyrimidine-1-yl)-1H-benzoimidazol-2-yl]-1H-pyridine-2-one; and

4-[2-(4-Methoxy-3-Chloro-phenyl)-2-(S)-hydroxy-ethylamino]-3-[4-methyl-6-(1,4,5,6-tetrahydropyrimidine-1-yl)-1H-benzoimidazol-2-yl]-1H-pyridine-2-one;

4-[2-(3-Chloro-4-methoxy-phenyl)-2-hydroxy-ethylamino]-3-(4-methyl-6-morpholin-4-yl-1H-benzoimidazol-2-yl)-1,5-dihydro-pyrrol-2-one;

4-[2-(3-Bromo-4-methoxy-phenyl)-2-hydroxy-ethylamino]-3-(4-methyl-6-morpholin-4-yl-1H-benzoimidazol-2-yl)-1,5-dihydro-pyrrol-2-one;

(S)-4-[2-(3-Chloro-phenyl)-2-hydroxy-ethylamino]-3-(4-methyl-6-morpholin-4-yl-1H-benzoimidazol-2-yl)-1,5-dihydro-pyrrol-2-one;

(S,S and S,R)-4-[2-(3-Chloro-phenyl)-2-hydroxy-ethylamino]-5-methyl-3-(4-methyl-6-morpholin-4-yl-1H-benzoimidazol-2-yl)-1,5-dihydro-pyrrol-2-one;

[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid tetrahydro-furan-3-ylmethyl ester;

[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid 2-methoxy-propyl ester;

(S)-2-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetamide Bis hydrochloride;

(S)-4-[2-(3-Chloro-phenyl)-2-hydroxy-ethylamino]-3-{6[4-(2-methyoxy-ethyl)-piperazin-1-yl]-4-methyl-1H-benzoimidazol-2-yl1H-pyridin-2-one bis-hydrochloride;

(S)-4-[2-(3-Bromo-phenyl)-2-hydroxy-ethylamino]-3-{6-[4-(2-methyoxy-ethyl)-piperazin-1-yl]-4-methyl-1H-benzoimidazol-2-yl}-1H-pyridin-2-one bis hydrochloride;

(S)-4-[2-(3-Cynao-phenyl)-2-hydroxy-ethylamino]-3-{6[4-(2-methyoxy-ethyl)-piperazin-1-yl]-4-methyl-1H-benzoimidazol-2-yl1H-pyridin-2-one bis hydrochloride;

(S)-4-[2-(3-Chloro-phenyl)-2-hydroxy-ethylamino]-3-{6-[4-(2-hydroxy-ethyl)-piperadin-1-yl]-4-methyl-1H-benzimidazol-2-yl}-1H-pyridin-2-one bis hydrochloride;

(S)-4-[2-(3-Chloro-phenyl)-2-hydroxy-ethylamino]-3-{4-methyl-6-[4-(2-methylsulfanyl-ethyl)-piperazin-1-yl]-1H-benzoimidazol-2-yl}-1H-pyridin-2-one bis hydrochloride;

(S)-4-[2-(3-Chloro-phenyl)-2-hydroxy-ethylamino]-3-[4-methyl-6-(3R-methyl-piperazin-1-yl)-1H-benzoimidazol-2-yl]-1H-pyridin-2-one bis hydrochloride; and

(S)₄-[2-(3-Chloro-phenyl)-2-hydroxy-ethylamino]-3-{6-[4-(2-methoxy-ethyl)-3(R)-methyl-piperazin-1-yl]-4-methyl-1H-benzoimidazol-2-yl}-1H-pyridin-2-one bis hydrochloride.

The IGF1R inhibitors of the present invention are useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

As used herein, the term “EGFR inhibitor” refers to any biological or small molecule that inhibits the activity of the EGF receptor, thereby providing an anti-cancer effect.

EGFR inhibitors that are biological molecules and are useful in the present invention include, for example, EGFR antibodies and functional equivalents thereof. Functional equivalents of antibodies have binding characteristics comparable to those of antibodies, and inhibit the growth of cells that express EGFR. In some embodiments, the EGFR inhibitor is cetuximab. In another embodiment of the present invention, the EGFR inibitor is erlotinib. In another embodiment of the present invention, the EGFR inhibitor is gefinitib. In another embodiment of the present invention, the EGFR inhibitor is ABX-EGF (Abgenix). In yet another embodiment, the EGFR inhibitor is EMD72000 (Merck KGA)

EGFR inhibitors that are small molecules and are useful in the present invention include, for example, the following:

U.S. Pat. No. 5,656,655 to Spada et al. discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted. The compounds disclosed in U.S. Pat. No. 5,656,655 are incorporated herein by reference.

U.S. Pat. No. 5,646,153 to Spada et al. discloses bis mono and/or bicyclic aryl heteroaryl, carbocyclic, and heterocarbocyclic compounds that inhibit EGFR. The compounds disclosed in U.S. Pat. No. 5,646,153 are incorporated herein by reference.

U.S. Pat. No. 5,679,683 to Bridges et al. discloses tricyclic pyrimidine compounds that inhibit the EGFR. The compounds are fused heterocyclic pyrimidine derivatives described at column 3, line 35 to column 5, line 6. The description of these compounds at column 3, line 35 to column 5, line 6 is incorporated herein by reference.

U.S. Pat. No. 5,616,582 to Barker discloses quinazoline derivatives that have receptor tyrosine kinase inhibitory activity. The compounds disclosed in U.S. Pat. No. 5,616,582 are incorporated herein by reference.

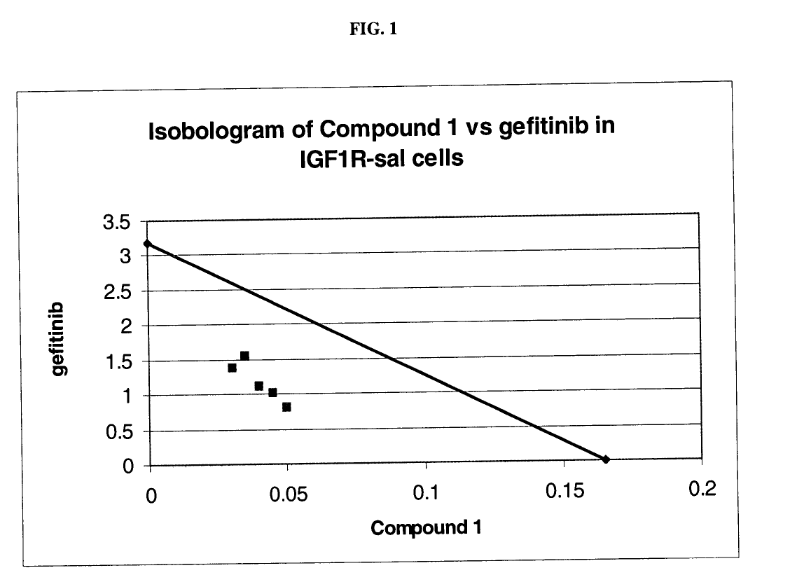

Fry et al., Science 265, 1093-1095 (1994) in FIG. 1 discloses a compound having a structure that inhibits EGFR. The compound shown in FIG. 1 of the Fry et al. article is incorporated herein by reference.

Osherov et al. disclose tyrphostins that inhibit EGFR/HER1. The compounds disclosed in the Osherov et al. article, and, in particular, those in Tables I, II, III, and IV are incorporated herein by reference.

U.S. Pat. No. 5,196,446 to Levitzki et al. discloses heteroarylethenediyl or heteroarylethendeiylaryl compounds that inhibit EGFR. The compounds disclosed in U.S. Pat. No. 5,196,446 from column 2, line 42 to column 3, line 40 are incorporated herein by reference.

Panek et al., Journal of Pharmacology and Experimental Therapeutics 283, 1433-1444 (1997) discloses a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436. The compound described in FIG. 1 on page 1436 of the Panek et al. article is incorporated herein by reference.

The present invention also encompasses a pharmaceutical composition useful in the treatment of cancer, comprising a therapeutically effective amount of the combinations of this invention and may comprise an additional anti-cancer agent or agents, and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like.

The IGF1R and EGFR inhibitors of the present invention may be administered orally or parenterally including the intravenous, intramuscular, intraperitoneal, subcutaneous, rectal and topical routes of administration.

For oral use, IGF1R and EGFR inhibitors and compositions of this invention may be administered, for example, in the form of tablets or capsules, powders, dispersible granules, or cachets, or as aqueous solutions or suspensions. In the case of tablets for oral use, carriers that are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added. In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations that are intended for conversion, shortly before use, to liquid preparations for either oral or parenteral administration. Such liquid forms include solutions, suspensions and emulsions.

The IGF1R and/or EGFR inhibitor may also be delivered transdermally. The transdermal compositions can take the form of creams, lotions, aerosols and/or emulsions and can be included in a transdermal patch of the matrix or reservoir type as are conventional in the art for this purpose.

The IGF1R inhibitor may be administered prior to, simultaneously with, or subsequent to the administration of the EGFR inhibitor.

The combinations of the present invention may also be used in conjunction with other well-known anticancer therapies, including radiation, chemotherapy and surgery. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

The actual dosage employed may be varied depending upon the requirements of the patient and the severity of the condition being treated. Generally, treatment is initiated with smaller dosages that are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Also, in general, the IGF1R inhibitor and the EGFR inhibitor do not have to be administered in the same pharmaceutical composition, and may, because of different physical and chemical characteristics, have to be administered by different routes. For example, the IGF1R inhibitor may be administered orally to generate and maintain good blood levels thereof, while the EGFR inhibitor may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of IGF1R inhibitor and EGFR inhibitor and/or radiation chemotherapy and/or surgery will depend upon the diagnosis of the attending physicians and their judgment of the condition of the patient and the appropriate treatment protocol.

Administration of either the EGFR inhibitor and/or the IGF1R inhibitor may be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

Thus, in accordance with experience and knowledge, the practicing physician can modify each protocol for the administration of a component (therapeutic agent—i.e., IGF1R inhibitor, EGFR inhibitor, additional anticancer drugs, surgery, or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In order to facilitate a further understanding of the invention, the following examples are presented primarily for the purpose of illustrating more specific details thereof. The scope of the invention should not be deemed limited by the examples, but encompasses the entire subject matter defined in the claims.

### EXAMPLE 1

³H-Thymidine Uptake Cell Proliferation Assay Utilizing Drug Combinations of IGF1R Inhibitors and EGFR Inhibitors

Stock drug concentrations were 10 mM in 100% DMSO (dimethyl sulfoxide), with subsequent dilutions performed in 70% DMSO.

Serial dilutions (1:4 or 1:5) were used to establish the 50% inhibitory dose of both the test and standard compounds alone. The cells were seeded in a 50 ul volume using a 96-well format 24 hrs prior to addition of the drug. The next day, each well received an additional 25 ul of the test compound or media (containing DMSO), and 25 ul of the standard compound or media (containing DMSO). A dose response curve was established for the standard compound; the test compound was then added as a single dose to the standard compound dose curves. All wells contain a final volume of 100 ul and a final concentration of 0.35% DMSO.

After dosing, the cells were allowed to incubate at 37° C. in an atmosphere of 5% CO₂ until they were labeled with 0.44 uCi/well ³H-thymidine; after a total of 72 hours post dosing, wells were harvested. Wells without cells were used to calculate a background value, and wells with cells but without drug were used to calculate a total control value. At harvest, the cells were trypsized and the amount of ³H-thymidine incorporated was captured by glass filter and counted by scintillation.

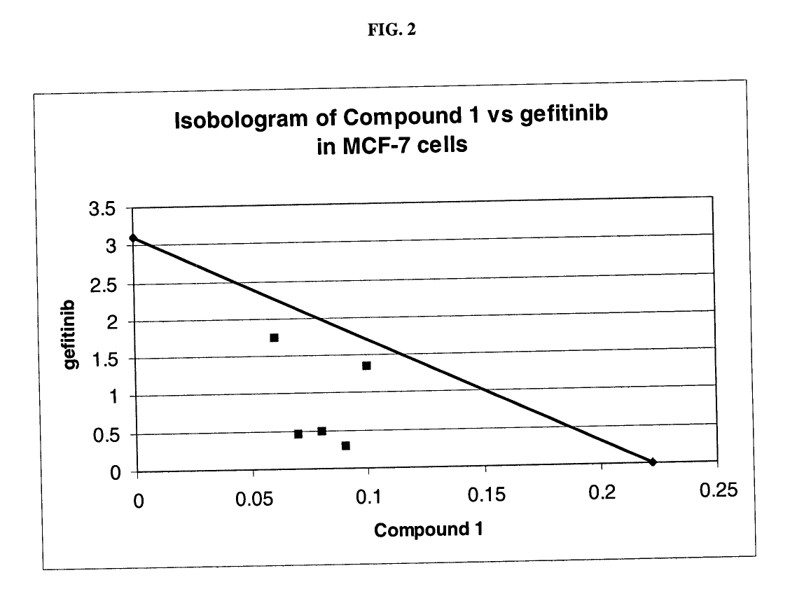

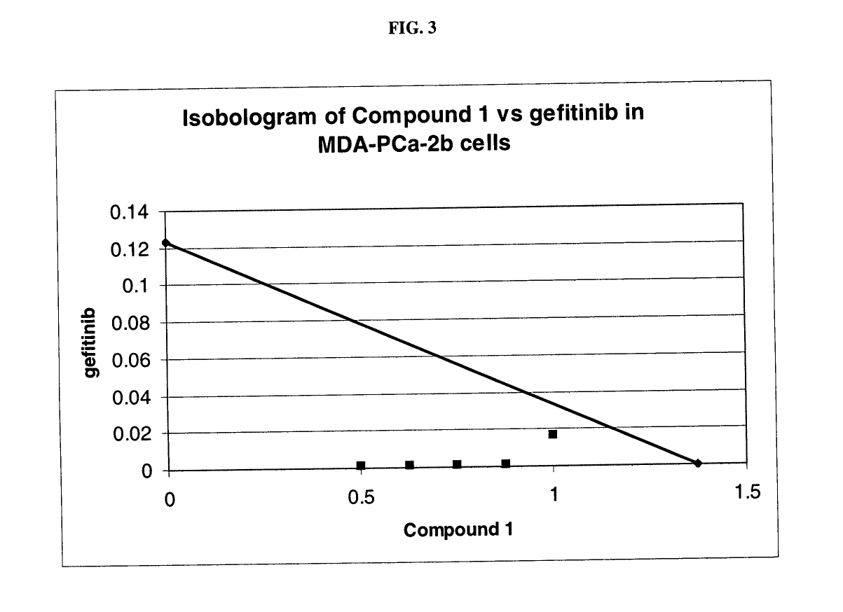

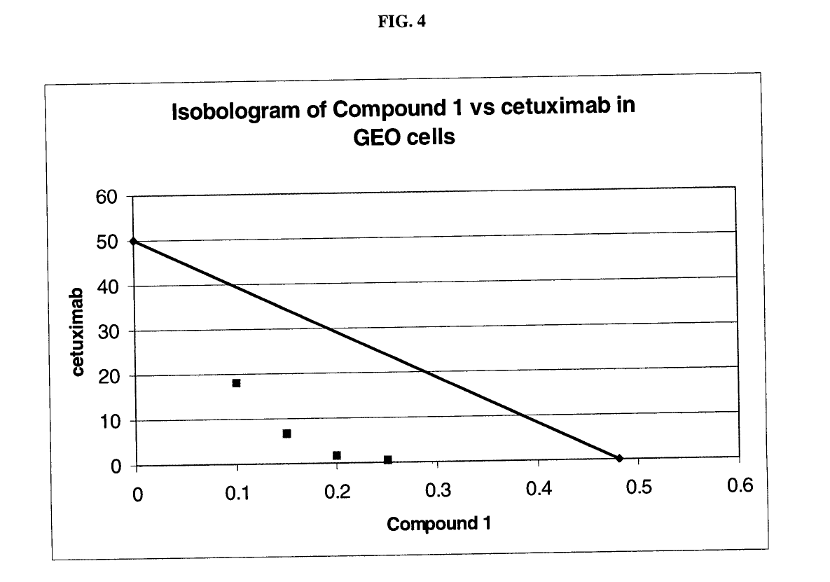

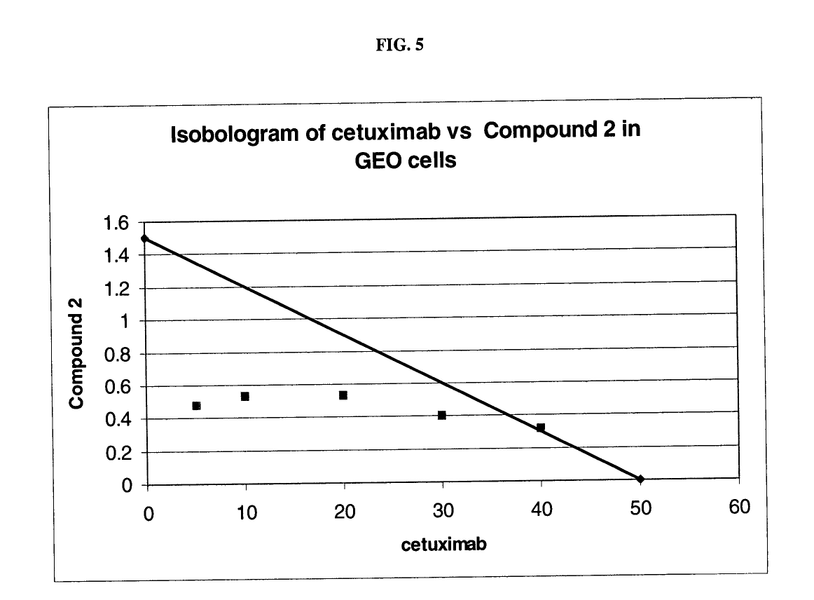

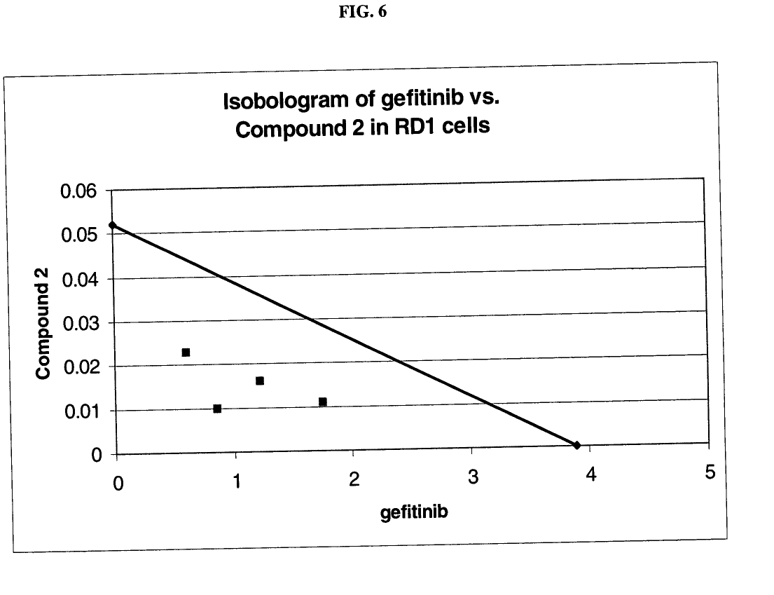

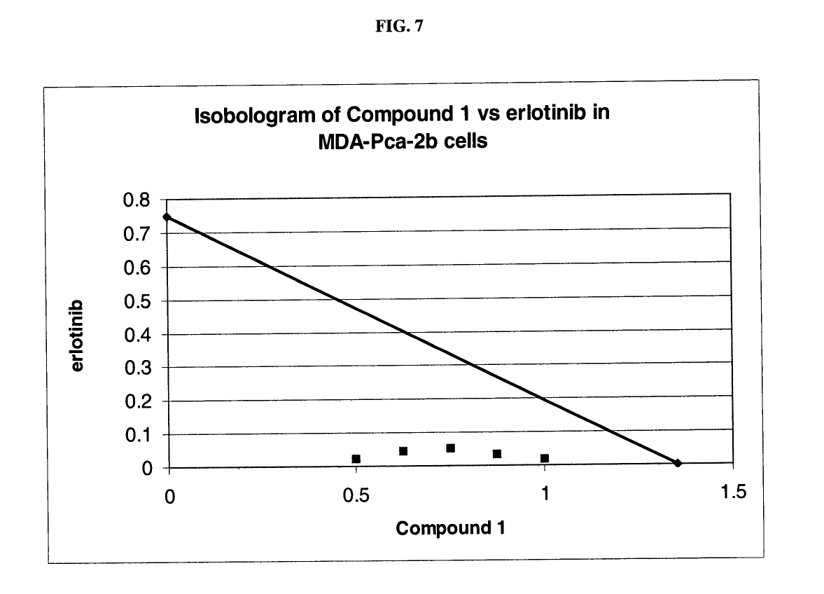

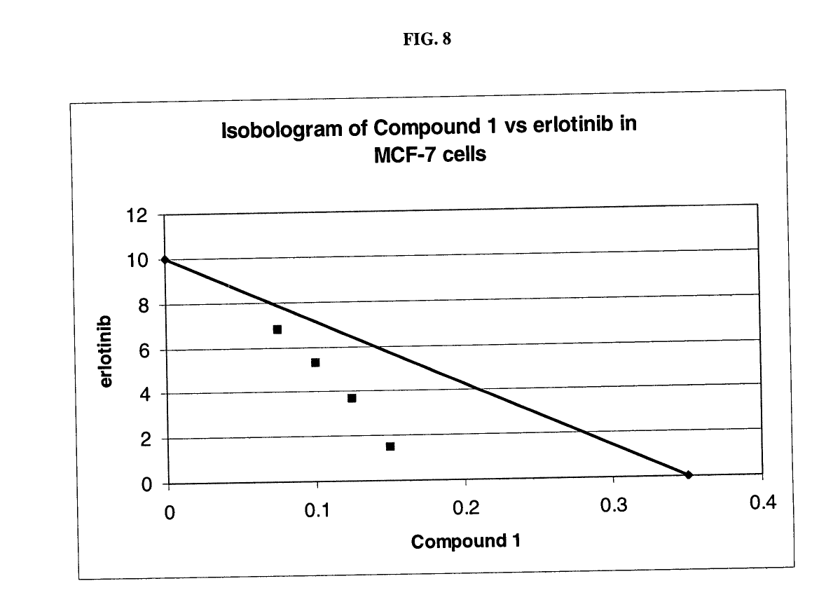

Concentrations of each drug alone or combinations of the two drugs administered together that blocked growth by 50% (IC₅₀) were calculated. Assuming zero interaction between the two compounds, these points on the axes can be joined by a straight line (isobole) which indicates combinations of standard and test drugs that are isoeffective with either drug alone. The isoeffect is the IC₅₀. When drug combinations fall along this straight line they are assumed to be additive. When the drug combinations are more effective than expected, lower concentrations are required to produce the isoeffect (IC₅₀) and are considered synergistic. These points will fall below the zero interaction isobole. When drug combinations require higher concentrations than expected to produce the isoeffect, they are considered antagonistic and the points will fall above the zero interaction isobole. All of the combinations tested fall at or below the zero interaction isobole as depicted in Figures 1 through 8 wherein “Compound 1” and “Compound 2” represent IGF1R inhibitors according to Formula I.

### EXAMPLE 2

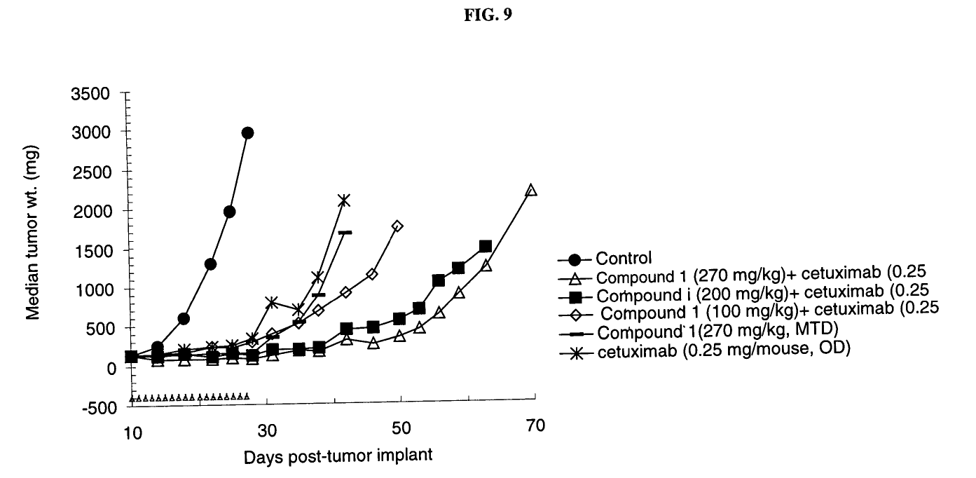

Chemotherapy trials were conducted with an IGF1R inhibitor (Compound 1) and an EGFR inhibitor (cetuximab), either singly or in combination, in nude mice bearing advanced-stage GEO human colon carcinoma xenografts. As monotherapy, both agents demonstrated significant antitumor activities, inhibiting tumor growth/progression and causing significant tumor growth delay (TGD, delay of tumor progression to a predetermined tumor burden). Treatment of mice with Compound 1 at its MTD of 270 mg/kg/adm, po, qdx17 yielded TGD value of 18.5 days. Cetuximab at its optimal dose of 0.25 mg/mouse, ip, q3dx6, produced TGD of 14.5 days. However, when used in combination the two agents produced antitumor efficacies that were far superior than those that could be produced maximally by either single agent alone (i.e., at their MTD or OD). Thus, using the maximally tolerated regimen (270 mg/kg/adm Compound 1 plus 0.25 mg/mouse cetuximab) the combination produced a TGD of 40.3 days, significantly better than single agent Compound 1 (P=0.0009) or single agent cetuximab (P=0.0008). Even more significant, superior antitumor efficacies were obtained with combination regimens that were below the maximally tolerated level and thus effectively improving the efficacy/tolerability margin of therapeutic strategies that target EGFR and IGF1R for the treatment of cancer. FIG. 9 depicts the effects of Compound 1 and cetuximab treatment, singly and in combination, on the growth of the GEO human colon carcinoma xenograft model in nude mice.

Importantly, in this study several combination regimens of Compound 1 and cetuximab, even at dose levels that are clearly below the MTD level, produced antitumor efficacies that were significantly superior than the optimal efficacy obtained with either single agent alone (at their respective MTD or OD), thus satisfying the definition of therapeutic synergism. On the other hand, the combination of Compound 1 and cetuximab produced toxicity that was no greater than either single agent alone, in terms of both weight loss and mortality.

The present invention is not limited to the embodiments specifically described above, but is capable of variation and modification without departure from the scope of the appended claims.

We claim:

1. A method for the synergistic treatment of cancer comprising administering to a mammal in need thereof a therapeutically effective amount of an EGFR inhibitor in combination with a therapeutically effective amount of an IGF1R inhibitor in amounts sufficient to achieve synergistic effects.

2. The method according to claim 1 wherein said EGFR inhibitor is cetuximab.

3. The method according to claim 1 wherein said EGFR inhibitor is erlotinib.

4. The method according to claim 1 wherein said EGFR inhibitor is gefitinib.

5. The method according to claim 1 wherein said EGFR inhibitor is EKB-569.

6. The method according to claim 1 where in said EGFR inhibitor is ABX-EGF.

7. The method according to claim 1 wherein said IGF1R inhibitor has the following formula I 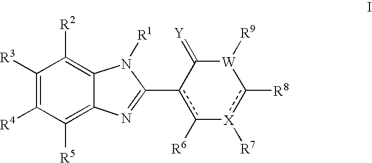
its enantiomers, diastereomers, pharmaceutically acceptable salts, hydrates, prodrugs and solvates thereof;
wherein
X is N, C₁-C₃ alkyl, or a direct bond;
Y is O or S;
W is N, C, O, or S; provided that if W is O or S, R⁹ is absent;
R¹ is H, alkyl, or alkoxy;
R² and R⁹ are independently H or alkyl;
R³ is H, C₁₋₆ alkyl, alkenyl, alkynyl, cycloalkyl, heterocycloalkyl, halo, amino, —OR⁶⁰, —NO₂, —OH, —SR⁶⁰, —NR⁶⁰R⁶¹, —CN, —C(O)R⁶⁰, —CO₂R⁶⁰, —CONR⁶¹R⁶¹, OCONR⁶⁰R⁶¹, —NR⁶²CONR⁶⁰R⁶¹, —NR⁶⁰SO₂R⁶¹, −SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3:
R⁴ is H, halo, alkyl or haloalkyl;
R⁵ is H, alkyl, halo, or aryl;
R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;
R⁶⁰, R⁶¹, R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;
R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹, heteroaryl or heterocycloalkyl; and
R³⁰ and R³¹ are, independently, hydrogen, alkyl, or cycloalkyl.

8. The method of claim 6 wherein R³ is an optionally substituted morpholine, thiomorpholine, sulfoxymorpholine, sulfonylmorpholine, or homomorpholine.

9. The method according to claim 6 wherein R³ is a substituted or unsubstituted piperazine or piperadine.

10. The method according to claim 6 wherein R⁶ is —NH-Z-aryl, or —NH-Z-heteroaryl.

11. The method of claim 9 wherein said aryl is a substituted or unsubstituted phenyl.

12. The method of claim 9 wherein said heteroaryl is a substituted or unsubstituted pyridinyl, imidazolyl, pyrazolyl, pyrrolyl or triazolyl.

13. The method of claim 1 wherein the EGFR inhibitor is cetuximab and the IGF1R inhibitor is selected from the group consisting of:
(±)-4-[2-(3-Chloro-4-fluoro-phenyl)-2-hydroxy-ethylamino]-3-(6-imidazol-1-yl-4-methyl-1H-benzimidazol-2-yl)-1H-pyridin-2-one;
(S)-4-[2-(3-Fluoro-phenyl)-1-hydroxymethyl-ethylamino]-3-(6-imidazol-1-yl-4-methyl-1H-benzimidazol-2-yl)-1H-pyridin-2-one;
(±)-4-[2-(3-Chloro-phenyl)-2-hydroxy-ethylamino]-3-(6-imidazol-1-yl-1H-benzimidazol-2-yl)-1H-pyridin-2-one;
(S)-4-[2-(3-Chloro-phenyl)-2-hydroxy-ethylamino]-3-(4-methyl-6-morpholin-4-yl-1H-benzoimidazol-2-yl)-1H-pyridin-2-one;
(S)-2-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetamide Bis hydrochloride;
(S)-4-[2-(3-Chloro-phenyl)-2-hydroxy-ethylamino]-3-{4-methyl-6-[4-(2-methylsulfanyl-ethyl)-piperazin-1-yl]-1H-benzoimidazol-2-yl}-1H-pyridin-2-one bis hydrochloride;
(S)₄-[2-(3-Chloro-phenyl)-2-hydroxy-ethylamino]-3-[4-methyl-6-(3R-methyl-piperazin-1-yl)-1H-benzoimidazol-2-yl]-1H-pyridin-2-one bis hydrochloride; and
(S)-4-[2-(3-Chloro-phenyl)-2-methoxy-ethylamino]-3-{6-[4-(2-hydroxy-ethyl)-piperazin-1-yl]-4-methyl-1H-benzimidazol-2-yl}-1H-pyridin-2-one.
